(19) United States
(12) Patent Application Publication
(10) Pub. No.: US 2021/0353791 A1
(43) Pub. Date: Nov. 18, 2021

(54) SYSTEM FOR NEUTRALIZING BIOLOGICAL ORGANISMS

(71) Applicant: Ensign-Bickford Aerospace & Defense Company, Simsbury, CT (US)

(72) Inventors: Richard M. Kellett, Southwick, MA (US); Alan J. Garvey, Suffield, CT (US); Breck O. Parker, Saco, ME (US)

(21) Appl. No.: 17/308,475

(22) Filed: May 5, 2021

Related U.S. Application Data
(60) Provisional application No. 63/026,332, filed on May 18, 2020.

(51) Int. Cl. A61L 2/06; A61L 2/20; A61L 2/26

(52) U.S. Cl. CPC A61L 2/06; A61L 2/204; A61L 2202/123; A61L 2202/121; A61L 2202/122; A61L 2/26

(57) ABSTRACT

A system and method for neutralizing a biological organism is provided. The system includes a first pyrotechnic element. A first element is made from decontamination material, the decontamination material decomposing into a decontamination gas in response to thermal energy from the first pyrotechnic element. a selectively sealable chamber is fluidly coupled to the first element to receive the decontamination gas, the chamber being sized to receive an article.

### CROSS REFERENCE TO RELATED APPLICATIONS

This application claims the benefit of U.S. Provisional Application Ser. No. 63/026,332 filed May 18, 2020, the entire disclosure of which is incorporated herein by reference.

### BACKGROUND

The subject matter disclosed herein relates to a system for delivering a material that neutralizes biological organisms, and in particular to a system that gasifies a material to neutralize biological threats and disinfect articles such as equipment, clothing or personal protective equipment.

Biological threats or agents are organisms (including viruses) that may cause harm or sickness to humans or other mammals. Some of these organisms, such as Bacillus anthracis (i.e. anthrax) have been modified such that they may be delivered in a concentrated form in an attempt to cause harm. Others, such as severe acute respiratory syndrome corona virus 2 (e.g. SARS-CoV-2 or Covid-19), may be a naturally occurring mutation for which humans or animals may not have a natural immunity. When such an organism spreads or is distributed, the area in which the release takes place may result in personnel wearing personal protective equipment (PPE), such as gloves, masks, eye protection, or clothing for example. Cleaning or decontamination is difficult because these organisms, some of which are bacterial endospores or viruses, may be resistant to treatments such as heat, desiccation, radiation, pressure and chemicals. Further, the organism may remain viable on surfaces or within fibers for hours, if not days.

Typically, PPE is intended to be a single use item. Meaning the article is disposed of immediately after use. Decontamination of these articles can be tenuous as typically the process uses bleach or heat or some other topical disinfectant, which can deteriorate the material's protective qualities. Further, when additional new PPE is not available, the personnel may reuse contaminated PPE or forego the use of PPE altogether. Where other articles, such as clothing or equipment for example, are contaminated, it may be difficult to provide a desired level of decontamination. This again may result in contaminated articles being used.

Accordingly, while existing systems and methods of decontaminating articles are suitable for their intended purposes the need for improvement remains, particularly in providing a system that can decontaminate articles for reuse that contains the decontamination material in a stable state and can selectively transform the decontamination material into a gaseous form to neutralize the biological threat.

### BRIEF DESCRIPTION

According to one aspect of the disclosure a system for neutralizing a biological organism is provided. The system includes a first pyrotechnic element. A first element is made from decontamination material, the decontamination material decomposing into a decontamination gas in response to thermal energy from the first pyrotechnic element. a selectively sealable chamber is fluidly coupled to the first element to receive the decontamination gas, the chamber being sized to receive an article.

In addition to one or more of the features described herein, or as an alternative, further embodiments of the system may include the decontamination material being paraformaldehyde. In addition to one or more of the features described herein, or as an alternative, further embodiments of the system may include an exhaust conduit fluidly coupled to the chamber, the exhaust conduit having an end exposed to an environment. In addition to one or more of the features described herein, or as an alternative, further embodiments of the system may include a valve disposed in fluid communication between the chamber and the end, the valve being configured to move between a closed position and an open position.

In addition to one or more of the features described herein, or as an alternative, further embodiments of the system may include a means for determining a parameter associated with the decontamination gas in the chamber, where the valve is configured to open in response to the means determining the parameter is equal to or greater than a threshold. In addition to one or more of the features described herein, or as an alternative, further embodiments of the system may include the means for determining the parameter having a controller and a sensor, the controller being electrically coupled to the valve and the sensor. In addition to one or more of the features described herein, or as an alternative, further embodiments of the system may include the parameter having a residence time of the decontamination gas in the chamber, and the controller is operable to measure the residence time.

In addition to one or more of the features described herein, or as an alternative, further embodiments of the system may include the parameter having a concentration level of the decontamination gas in the chamber, and the controller is operable to measure the concentration level. In addition to one or more of the features described herein, or as an alternative, further embodiments of the system may include a first cartridge fluidly and removably coupled to the chamber, the first pyrotechnic element and the first element being disposed within the first cartridge.

In addition to one or more of the features described herein, or as an alternative, further embodiments of the system may include a second pyrotechnic element and a second element selectively fluidly coupled to the chamber, the second element being made from a neutralization material, the neutralization material decomposing into a neutralization gas in response to thermal energy from the second pyrotechnic element. In addition to one or more of the features described herein, or as an alternative, further embodiments of the system may include the neutralization material having one of ammonium carbonate, ammonium bicarbonate, or a mixture of calcium oxide and ammonium chloride. In addition to one or more of the features described herein, or as an alternative, further embodiments of the system may include a second cartridge fluidly and removably coupled to the chamber, the second pyrotechnic element and the second element being disposed within the second cartridge.

In addition to one or more of the features described herein, or as an alternative, further embodiments of the system may include the parameter being a concentration level of the neutralization gas in the chamber, and the controller is operable to measure the concentration level. In addition to one or more of the features described herein, or as an alternative, further embodiments of the system may include a lid coupled to the chamber, the lid selectively sealing the chamber from an external environment.

According to another aspect of the disclosure, a method for neutralizing a biological organism on an article is provided. The method includes activating a first pyrotechnic element. Thermal energy is transferred from the first pyrotechnic element to a decontamination material. The decontamination material is decomposed into a decontamination gas in response to the thermal energy from the first pyrotechnic element. The decontamination gas flows into a chamber containing the article contaminated with the biological organism.

In addition to one or more of the features described herein, or as an alternative, further embodiments of the method may include placing an article containing the biological organism into the chamber prior to activating the first pyrotechnic element. In addition to one or more of the features described herein, or as an alternative, further embodiments of the method may include determining a parameter associated with the decontamination gas and the biological organism is equal to or exceeds a predetermined threshold, and exhausting the decontamination gas from the chamber when the parameter is equal to or exceeds the predetermined threshold.

In addition to one or more of the features described herein, or as an alternative, further embodiments of the method may include the parameter being at least one of a residence time of the decontamination gas in the chamber, or a concentration level of the decontamination gas within the chamber. In addition to one or more of the features described herein, or as an alternative, further embodiments of the method may include activating a second pyrotechnic element; transferring thermal energy from the second pyrotechnic element to a neutralization material; decomposing the neutralization material into a neutralization gas in response to the thermal energy from the second pyrotechnic element; and flowing the neutralization gas into the chamber containing the decontamination gas.

In addition to one or more of the features described herein, or as an alternative, further embodiments of the method may include reacting the decontamination gas with neutralization gas to form a third compound. In addition to one or more of the features described herein, or as an alternative, further embodiments of the method may include the decontamination gas being formaldehyde, the neutralization gas being ammonia, and the third compound is hexamethylene tetramine. In addition to one or more of the features described herein, or as an alternative, further embodiments of the method may include waiting a predetermined amount of time with the decontamination gas in the chamber before exhausting the decontamination gas. In addition to one or more of the features described herein, or as an alternative, further embodiments of the method may include waiting until a predetermined concentration level of the neutralization gas is less than a threshold within the chamber before exhausting the gaseous contents of the chamber.

These and other advantages and features will become more apparent from the following description taken in conjunction with the drawings.

### BRIEF DESCRIPTION OF DRAWINGS

The subject matter, which is regarded as the disclosure, is particularly pointed out and distinctly claimed in the claims at the conclusion of the specification. The foregoing and other features, and advantages of the disclosure are apparent from the following detailed description taken in conjunction with the accompanying drawings in which:

FIG. 1 is a block diagram of a decontamination system in accordance with an embodiment;

FIG. 2 is a block diagram of a decontamination system in accordance with another embodiment; and

FIG. 3 is side view of a mortar shell for delivering the decontamination system in accordance with an embodiment of the invention.

The detailed description explains embodiments of the disclosure, together with advantages and features, by way of example with reference to the drawings.

### DETAILED DESCRIPTION

Embodiments of the present invention provide advantages in allowing the decontamination of an area containing a biological threat. Embodiments of the invention provide a decontamination device that includes a chemically stable decontamination material that may be selectively gasified to neutralize a biological threat. Embodiments of the invention provide a decontamination device that may be delivered to a remote location and activated to neutralize a biological threat. Embodiments of the invention further provide a decontamination device that may be delivered via an aerial vehicle. Embodiments of the invention provide a decontamination device that may be rechargeable with a chemically stable decontamination material that may be selectively gasified to neutralize a biological threat for reuse.

Biological threats pose a hazard to humans and an area contaminated with such an organism needs to be neutralized before being suitable for occupation. As used herein the term “biological threat” refers to a biological organism that may be deployed to produce casualties in personnel or animals or damage plants. Organisms, include, but are not limited to, unicellular and multicellular microorganisms such as bacteria, bacterial spores, yeast, molds, fungi, fungal spores, viruses, viral particles, parasites, and the like Examples of a biological threat include viruses, such as SARS-CoV-2 for example, and bacterial endospores, such as Bacillus anthracis (i.e. anthrax), Bacillus subtilis and Encephalomyelitis for example.

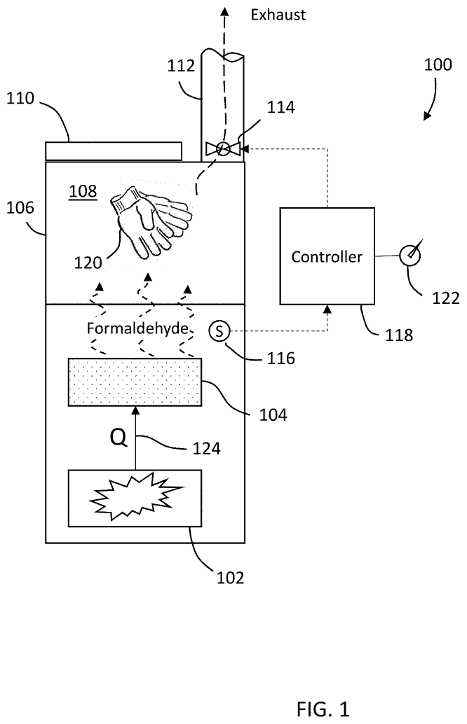

Referring now to FIG. 1, an embodiment is shown of a decontamination system 100. The decontamination system 100 includes a thermal generator 102 thermally connected with a decontamination material module 104. The thermal generator 102 may be a pyrotechnic element that is thermally stable until activated. In an embodiment, the thermal generator 102 may include an initiator and a transfer charge that selectively activates the pyrotechnic element. The initiator may be a device or means such as but not limited to a pin pull detonator, or an electrically powered heated bridge wire for example. Where the initiator is electrically powered, electrical power source may be a battery. The transfer charge may be a mixture of iron oxide and zirconium metal, or a mixture of lead-oxide and elemental silicon for example.

In an embodiment, the decontamination material module 104 may include a material such as paraformaldehyde. In an embodiment, the paraformaldehyde is in the form of a slab, compressed particulates, or as a cake held together with a non-interfering binder, such as polyethylene carbonate or analogous carbon dioxide based polymers. It should be appreciated that while the decontamination material module is described herein as including paraformaldehyde, this is for example purposes and the claims should not be so limited. In other embodiments, other thermally activated decontamination materials may be used.

In an embodiment, the thermal generator 102 and the decontamination material module 204 are removable or replaceable to allow the system 100 to be operated again after the pyrotechnic element or decomposition material have been depleted.

In the embodiment of FIG. 1, the decontamination material module 104 is gaseously or fluidly coupled to a chamber 106. The chamber 106 includes a housing that defines a hollow interior space 108. The interior space 108 is sized to fit the articles 120 to be decontaminated, such as PPE, clothing or equipment for example. The chamber 106 includes a lid or door 110 that seals the interior space 108 from the environment. An exhaust conduit 112 is fluidly coupled to the interior space 108. In an embodiment, the exhaust conduit 112 is selectively opened via a valve 114. The valve 114 being movable between a closed position (i.e. chamber sealed) and an open position (i.e. exhausting gas to the external environment). In an embodiment, the conduit 112 may include a neutralizing agent such as ammonia for example that is injected into the exhaust stream. The injection of the neutralizing agent reacts with the decontamination gas to generate a third compound. In still another embodiment, the exhaust conduit 112 may include a filter. In an embodiment, the filter may include an absorbent, such as activated charcoal or a neutralizing agent for example, that the exhaust gas passes through before entering the environment.

In an embodiment, a sensor 116 is operably arranged to measure a parameter of the system 100. In an embodiment, the sensor 116 is a temperature sensor, such as a thermocouple for example, that measures the temperature within the interior space 108 or the housing of the decontamination material module 104 for example. It should be appreciated that in other embodiments, other sensors 116 may be used, such as a sensor that detects the activation of the initiator for example. The sensor 116 outputs a signal to a controller 118, the signal being an indication of the parameter. When the parameter is equal to or exceeds a threshold, such as when the temperature exceeds the temperature where the decontamination material (e.g. paraformaldehyde) decomposes for example, the controller 118 initiates a timer. Since decontamination of the article 120 may depend on the amount of time to which it has been exposed to the gas (e.g. formaldehyde) generated by the decontamination material module 104. In some embodiments, the decontamination process may wait a predetermined amount of time before exhausting the decontamination gas. It should be appreciated that in other embodiments, other operating parameters may be used. For example, in other embodiments, the sensor 116 may detect the concentration level of gaseous decontamination material within the chamber 106.

When the operating parameter exceeds a threshold (e.g. time), the controller activates the valve 114, to allow the gaseous decontamination material within the chamber 106 to be exhausted via conduit 112. In an embodiment, the conduit 112 may include an air movement device, such as a fan for example, to facilitate evacuation of the gaseous decontamination material. In an embodiment, the chamber 106 may include an air inlet, such as a one-way or check valve for example, that allows replacement air to entire the chamber 106 when the decontamination gas is exhausted via conduit 112.

In an embodiment, the controller 118 may include a user interface 122 that allows the operator to control the operation of the system 100. In an embodiment, the user interface 122 includes a selector that allows the operator to select the type of biological threat the operator would like to neutralize. For example, the operator may select between a virus (e.g. SARS-CoV-2) or a bacterial endospore (e.g. anthrax). Based at least in part on the selection by the operator, the controller 118 may change control methods, such as but not limited to the length of time or the concentration level that the article 120 is exposed to the gaseous decontamination material.

In an embodiment, the user interface 122 may include visual indicators (e.g. light emitting diodes) that indicate when the operating parameter (e.g. time or concentration level) has been exceeded. In an embodiment, the operator may manually open the valve 114 in response to the visual indicator being activated.

In operation, the operator places the articles 120 into the interior space 108 of chamber 106. After closing and sealing the lid or door 110, the operator activates the initiator, such as by pulling a pin or via the user interface 122 for example. The initiator activates the transfer charge which applies heat to the pyrotechnic element. Accordingly, heat is liberated with resulting propagation in a self-sustaining manner throughout the entire thermal generator 102 in a controlled manner. In an embodiment, the lid or door 110 may be locked to prevent opening while the decontamination gas is present within the chamber 106. In an embodiment, the lock may be an interlock device that allows the door 110 to be opened based on a parameter, such as amount of time the decontamination gas has been exhausted, or based on a concentration level of the decontamination gas in the chamber 106. In still other embodiments, the lock for the door 110 may include an interlock that prevents the flowing of decontamination gas, or the activation of the pyrotechnic element unless the door 110 is closed and locked.

In some embodiments, the exothermic reaction between the dissimilar materials that comprise the pyrotechnic element can be made to occur at a relatively slower propagation or burn rate in part not only due to the composition of the dissimilar materials selected but also due to the three-dimensional characteristics of the structure; in particular, to a non-uniform and varying distribution of the mass of the substrate and corresponding coating along the direction of the primary propagation or burn path. As is discussed herein, the burn rate and the amount of thermal energy released by the pyrotechnic element is used to cause a phase change in a decontamination material allowing the decontamination material to change into a gaseous state and flow into the interior space 108 and neutralize a biological threat.

The heat 124 from the thermal generator 102 is transferred to the decontamination material module 104. In the exemplary embodiment, the decontamination material within the module 104 is made from paraformaldehyde. Paraformaldehyde is a generally white crystalline solid having a density of about 1.42 g/cm³ and a melting point of 120° C. Paraformaldehyde is the smallest polyoxymethylene, the polymerization product of formaldehyde with a typical degree of polymerization of 8-100 units. The paraformaldehyde may be depolymerized into formaldehyde gas by heating, such as by the thermal energy released during the exothermic and alloying reaction of the thermal generator 102. The gasification of the paraformaldehyde into formaldehyde may then be released into the interior space 108. It should be appreciated that since paraformaldehyde is a stable material at normal environmental temperatures, advantages are gained in ease of storage, transportation and handling of the decontamination systems 100 prior to use. It has been found that biological threats, such as Bacillus anthracis (i.e. anthrax) for example, may be neutralized by fumigation with formaldehyde.

In an embodiment, the decontamination material (e.g. paraformaldehyde) may form a precipitate within the chamber 106. In these embodiments, the chamber 106 may include interior structures, such as but not limited to walls and shelves for example, that are heated to avoid having decontamination material within the chamber that has not gone through a phase change into the decontamination gas.

In an embodiment, in response to the operation of the thermal generator 102, the controller initiates a control method, such as a period of time for example. The control method ensures that the released formaldehyde gas has sufficient residence time within the interior space 108 to decontaminate the article 120. In an embodiment, the interior space may include an air movement device (e.g. a fan) or a mechanism for moving the article 120 while the formaldehyde gas is within the interior space 108.

Once the operating parameter exceeds a threshold, such as when the residence time or concentration levels have been achieved, the valve 114 is opened, such as in response to a signal from the controller 118. Once the formaldehyde gas has been exhausted or vented from the interior space 108, the operator may open the door 110 and remove the decontaminated articles 108. It should be appreciated that the decontaminated articles may be reused by personnel.

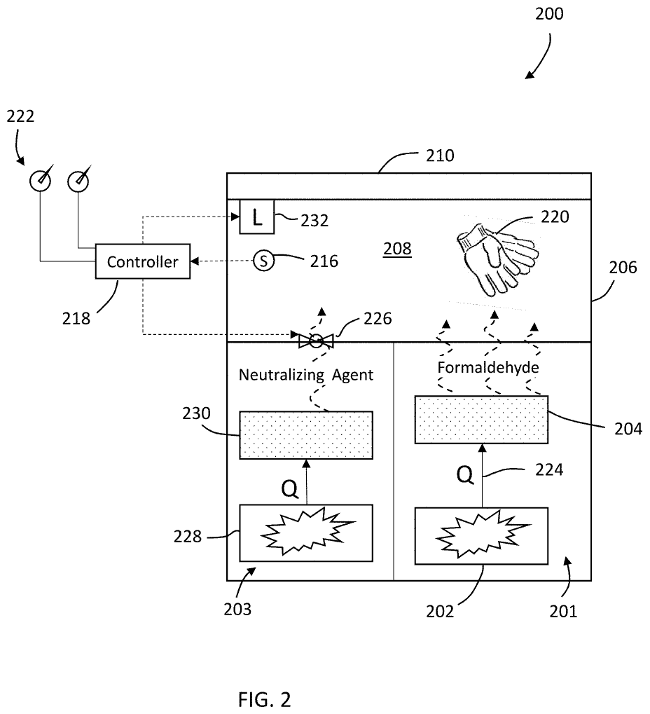

Referring now to FIG. 2, another embodiment is shown of a decontamination system 200. It should be appreciated that in some embodiments it may not be desirable or feasible to have a conduit for exhausting the decontamination gas, such as formaldehyde. Further, it may be undesirable to expose the operator to the decontamination gas. In the embodiment of FIG. 2, the system 200 includes a generator module 201 and a neutralizing module 203. The generator module 201 is similar to the system 100 in that it includes a thermal generator 202 and a decontamination material module 204.

In a similar manner to system 100, the thermal generator 202 includes a pyrotechnic element that generates thermal energy/heat that is transferred to a decontamination material in the decontamination material module. The decontamination material (e.g. paraformaldehyde) decomposes when exposed to thermal energy 224. The decontamination material module 204 is fluidly connected to the interior space 208 of a chamber 206 where the article 220 are placed to be decontaminated. A door 210 encloses and seals the interior space 208 from exterior environment.

In the embodiment of FIG. 2, the neutralizing module 203 is selectively fluidly coupled to the interior space 208, such as via valve 226 for example. The neutralizing module 203 includes a second thermal generator 228 and a neutralizing material module 230. The second thermal generator 228 may be constructed similar to thermal generators 102, 202 and include an initiator, a transfer charge, and a pyrotechnic element. The pyrotechnic element used in the second thermal generator 228 is suitable to generate sufficient heat to decompose a neutralizing material in the neutralizing material module 230. In an embodiment, the thermally decomposed neutralizing material generates ammonia gas. In an embodiment, the neutralizing material may be ammonium carbonate, ammonium bicarbonate, or a mixture of calcium oxide and ammonium chloride for example. The gaseous ammonia reacts with residual formaldehyde in the interior space 208 to form a third compound, such as hexamethylene tetramine for example. It should be appreciated that the operator may be exposed to the hexamethylene tetramine.

In an embodiment, the generator module 201 is isolated from the interior space 208 when the neutralizing material module 230 is generating a neutralizing gas. The isolation of the generator module 201 may be via a valve or a shutter for example. In an embodiment, the means used for isolating the generator module 201 seals (e.g. prevents gas flow) from the interior space 208 into the generator module 201.

In an embodiment, the neutralizing module 203 may include an alternate method for neutralizing the decontamination gas. In an embodiment, the neutralizing module may include a filter, such as but not limited to activated charcoal, a neutralizing agent, or a combination thereof for example. In an embodiment, the neutralizing module 203 may include an air movement device, such as a fan, that draws the air (with decontamination gas) from the interior space 208 and flows it through the filter. The filtered air is then flowed back into the interior space 208, or exhausted to the environment.

In an embodiment, the system 200 includes a controller 218 that is electrically coupled to a sensor 216 that measures a parameter of the system 200, such as temperature, decontamination gas or neutralization gas concentration levels for example. The controller 218 is further electrically coupled to the valve 226. The controller includes a user interface 222 allows the operator to control the operation of the system 200. In an embodiment, the user interface 222 allows the operator to select the type of decontamination material and neutralizing material that will be used. In another embodiment, the user interface 222 allows the operator to select the type of biological hazard or organism that may be present and the controller 218 determines the decontamination material and associated neutralizing material to be used in operation. The controller 218 may optionally be coupled to a door latch 232. As will be discussed in more detail, the latch 232 may be used to keep the door 210 closed and sealed until the decontamination gas has been neutralized.

In operation, the operator places the articles 220 into the interior space 208 of chamber 206 and closes the door 210. The thermal generator 202 is then activated, such as by activating the initiator for example. The activation of the thermal generator 202 may be via the user interface 222, or be manually activated by the operator. As discussed above with respect to FIG. 1, the activation of the thermal generator 202 results in the pyrotechnic element generating thermal energy 224 that is transferred to the decontamination module 204. The thermal energy 224 decomposes the decontamination material causing a decontamination gas to flow into the interior space 208 and into contact with the articles 220.

In an embodiment, the controller 218 determines the activation of the thermal generator 202, such as by an operator input with the user interface 222, or by a measurement of a parameter with a sensor 216 (e.g. a temperature sensor). Once the articles 220 have been exposed to the formaldehyde gas for a predetermined amount of time, the controller 218 activates the second thermal generator 228. The activation of the second thermal generator 228 decomposes the neutralizing material module 230 generating a neutralizing gas (e.g. ammonia). The controller 218 opens the valve 226 allowing the neutralizing gas to enter the interior space 208. In an embodiment that includes air management devices (e.g. fans or a filter), the controller 218 may activate the air management devices to provide a desired neutralization or exhausting of the air within the interior space 208.

Once the neutralizing gas has been resident in the interior space 208 for a predetermined amount of time, wherein the decontamination gas (e.g. formaldehyde) is neutralized. Once neutralized, the controller 218 transmits a signal to latch 232 unlocking the door 210 allowing the operator to remove the articles 220. As discussed herein, in embodiments the door 210 may include interlocks that prevent the door from being opened before the decontamination gas is neutralized or exhausted. In further embodiments, the door 210 may include interlocks that prevent the generator module 201 from being activated unless the door 210 is closed and sealed.

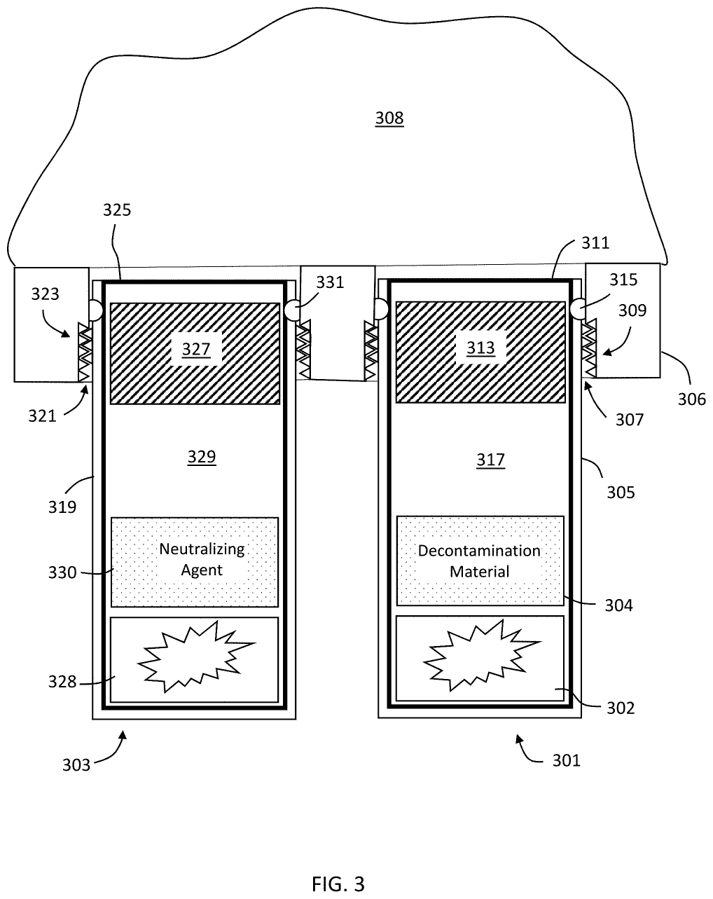

Referring now to FIG. 3, an embodiment of a decontamination system having replaceable decontamination cartridge 301 and optional neutralization cartridge 303. In this embodiment, the decontamination cartridge 301 includes a housing 305 with an attachment means, such as threads 307 that engage an attachment means 309 in a sidewall of the chamber 306. The housing 305 has an open end 311 that is enclosed by a plug 313. In an embodiment, the decontamination cartridge 301 includes a seal 315, between the housing 305 and a sidewall of the chamber 306.

The housing 305 defines a hollow interior 317 that contains a thermal generator 302 and a decontamination material module 304. The thermal generator 302 and the decontamination module 304 may be the same as the thermal generator 102 and the decontamination material module 104 described herein. In an embodiment, the plug 313 seals the open end 311 and prevents debris from entering the hollow interior 317. In an embodiment, the plug 313 is omitted and the decontamination material module 304 seals the end of the cartridge 301.

In an embodiment, an optional neutralization cartridge 303 may be provided. The neutralization cartridge 303 may be used in an application similar to FIG. 2, where an exhaust conduit is not available. The neutralization cartridge 303 includes a housing 319 having an attachment means, such as threads 321 for example, that couple with attachment means, such as threads 323 on the chamber 306. The housing 319 includes an open end 325 that is enclosed by a plug 327. In an embodiment the neutralization cartridge 303 includes a seal 331 that forms a seal between the housing 319 and the sidewall of the chamber 306.

The housing 319 includes a hollow interior 329. that contains a thermal generator 328 and a neutralizing material module 330. The thermal generator 328 and the neutralizing material module 330 may be the same as the thermal generator 228 and neutralizing material module 230 of FIG. 2. In an embodiment, the plug 327 seals the open end 325 and prevents debris from entering the hollow interior 329. In an embodiment, the plug 327 is omitted and the neutralization material module 330 seals the end of the cartridge 303.

In an embodiment, in a similar manner to the embodiment of FIG. 2, the neutralization cartridge 303 may include a filter instead of the thermal generator and the neutralizing agent is disposed within the filter. In this embodiment, the neutralizing agent may be a material that does not go through a phase change before neutralizing the decontamination gas. The neutralizing agent within the filter may include activated carbon, or a neutralizing material for example. In an embodiment, the cartridge 303 may include an air movement or air management device, such as a fan for example, that flows air (including decontamination gas) from the interior space 308 through the filter. In an embodiment, the air that passes through the filter is returned to the interior space 308. In another embodiment, the air that passes through the filter is exhausted to the environment. In an embodiment, the air is exhausted to the environment from an end of the cartridge 303 opposite the open end 325.

It should be appreciated that the thermal generators 302, 328 may be manually activated, such as by a pull pin for example, or may have an electrically activated initiator, such as an electrically powered heated bridge wire for example.

It should be appreciated that in some embodiments, the user interface 122, 222 may be omitted and the systems 100, 200 may be operated manually without the use of an electrical power source. This would provide advantages in allowing the systems 100, 200 to be used in a variety of applications and in the field without the operator having to be concerned about an energy source or having to recharge batteries. Further, the use of manually activated changeable cartridges 301, 303 would allow the systems 100, 200 to be operated as long as a supply of cartridges 301, 303 is available.

The term “about” is intended to include the degree of error associated with measurement of the particular quantity based upon the equipment available at the time of filing the application. For example, “about” can include a range of ±8% or 5%, or 2% of a given value.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

While the disclosure is provided in detail in connection with only a limited number of embodiments, it should be readily understood that the disclosure is not limited to such disclosed embodiments. Rather, the disclosure can be modified to incorporate any number of variations, alterations, substitutions or equivalent arrangements not heretofore described, but which are commensurate with the spirit and scope of the disclosure. Additionally, while various embodiments of the disclosure have been described, it is to be understood that the exemplary embodiment(s) may include only some of the described exemplary aspects. Accordingly, the disclosure is not to be seen as limited by the foregoing description, but is only limited by the scope of the appended claims.

What is claimed is:

1. A system for neutralizing a biological organism, the system comprising:
a first pyrotechnic element;
a first element made from decontamination material, the decontamination material decomposing into a decontamination gas in response to thermal energy from the first pyrotechnic element; and
a selectively sealable chamber fluidly coupled to the first element to receive the decontamination gas, the chamber being sized to receive an article.

2. The system of claim 1, wherein the decontamination material is paraformaldehyde.

3. The system of claim 1, further comprising an exhaust conduit fluidly coupled to the chamber, the exhaust conduit having an end exposed to an environment.

4. The system of claim 3, further comprising a valve disposed in fluid communication between the chamber and the end, the valve being configured to move between a closed position and an open position.

5. The system of claim 4, further comprising a means for determining a parameter associated with the decontamination gas in the chamber, where the valve is configured to open in response to the means determining the parameter is equal to or greater than a threshold.

6. The system of claim 5, wherein the means for determining the parameter includes a controller and a sensor, the controller being electrically coupled to the valve and the sensor.

7. The system of claim 6, wherein the parameter is a residence time of the decontamination gas in the chamber, and the controller is operable to measure the residence time.

8. The system of claim 6, wherein the parameter is a concentration level of the decontamination gas in the chamber, and the controller is operable to measure the concentration level.

9. The system of claim 1, further comprising a first cartridge fluidly and removably coupled to the chamber, the first pyrotechnic element and the first element being disposed within the first cartridge.

10. The system of claim 1, further comprising:
a second pyrotechnic element; and
a second element selectively fluidly coupled to the chamber, the second element being made from a neutralization material, the neutralization material decomposing into a neutralization gas in response to thermal energy from the second pyrotechnic element.

11. The system of claim 10, wherein the neutralization material is one of ammonium carbonate, ammonium bicarbonate, or a mixture of calcium oxide and ammonium chloride.

12. The system of claim 10, further comprising a second cartridge fluidly and removably coupled to the chamber, the second pyrotechnic element and the second element being disposed within the second cartridge.

13. The system of claim 10, wherein the parameter is a concentration level of the neutralization gas in the chamber, and the controller is operable to measure the concentration level.

14. The system of claim 1, further comprising a lid coupled to the chamber, the lid selectively sealing the chamber from an external environment.

15. A method for neutralizing a biological organism on an article, the method comprising:
activating a first pyrotechnic element;
transferring thermal energy from the first pyrotechnic element to a decontamination material;
decomposing the decontamination material into a decontamination gas in response to the thermal energy from the first pyrotechnic element; and
flowing the decontamination gas into a chamber containing the article contaminated with the biological organism.

16. The method of claim 15, further comprising placing an article containing the biological organism into the chamber prior to activating the first pyrotechnic element.

17. The method of claim 15, further comprising determining a parameter associated with the decontamination gas and the biological organism is equal to or exceeds a predetermined threshold, and exhausting the decontamination gas from the chamber when the parameter is equal to or exceeds the predetermined threshold.

18. The method of claim 17. wherein the parameter is at least one of a residence time of the decontamination gas in the chamber, or a concentration level of the decontamination gas within the chamber.

19. The method of claim 15, further comprising:
activating a second pyrotechnic element;
transferring thermal energy from the second pyrotechnic element to a neutralization material;
decomposing the neutralization material into a neutralization gas in response to the thermal energy from the second pyrotechnic element; and
flowing the neutralization gas into the chamber containing the decontamination gas.

20. The method of claim 19, further comprising reacting the decontamination gas with neutralization gas to form a third compound.

21. The method of claim 20, wherein the decontamination gas is formaldehyde, the neutralization gas is ammonia, and the third compound is hexamethylene tetramine.

22. The method of claim 15, further comprising waiting a predetermined amount of time with the decontamination gas in the chamber before exhausting the decontamination gas.

23. The method of claim 14, further comprising waiting until a predetermined concentration level of the neutralization gas is less than a threshold within the chamber before exhausting the gaseous contents of the chamber.
